(19) United States
(12) Patent Application Publication
(10) Pub. No.: US 2012/0058931 A1
(43) Pub. Date: Mar. 8, 2012

(54) Drug target that promotes secretory granule-granule fusion

(76) Inventors: Haruo Kasai, Tokyo (JP); Noriko Takahashi, Tokyo (JP); Vesa Olkkonen, Helskinki (FI); Herbert Y. Gaisano, Toronto (CA); Patrick Lam, Toronto (CA)

(21) Appl. No.: 12/796,035

(22) Filed: Sep. 3, 2010

(51) Int. Cl. A61K 38/00; G01N 33/566; C07K 2/00

(52) U.S. Cl. 514/1.1; 530/350; 436/501

(57) ABSTRACT

Munc18 proteins facilitate formation of SNARE complexes known to mediate membrane fusion. Much is known about the fusion of secretory granule (SG) to plasma membrane, mediated by Munc18a-SNARE complexes. It has been found that another Munc18 isoform, Munc18b, mediates SG-SG fusion which causes potentiation of secretion. The present invention has identified specific site mutations within Munc18b which further increased (called KR mutant) or reduced (called E59K mutant) SG-SG fusion compared to wild type (WT) Munc18b, causing amplification or reduction of insulin secretion, respectively. Compounds identified that mimic the actions of Munc18b-WT and Munc18b-KR mutant which increase SG-SG fusion, and those which mimic Munc18b-E59K mutant that block SG-SG fusion, are useful for treating and/or preventing diseases and/or conditions, whose underlying bases are a deficiency or excess of SG-SG fusion, respectively. These compounds also include conserved domains in Munc18a and Munc18c that mimic the KR and E59K sites in Munc18b.

In secretory cells, the membrane fusion machinery requires two key components: SNARE (soluble N-ethylmaleimide-sensitive factor attachment protein receptor) and SM (Sec1/Munc18) proteins. The SNARE paradigm dictates that cognate vesicle-associated (v-) SNAREs (Vesicle Associated Membrane Proteins, VAMPs, particularly VAMP2 and VAMP8) and target membrane-associated (t-) SNAREs (Syntaxins (Syn-1,2,3, and 4) and Synaptosome-Associated Protein of 25 kDa, SNAP-25) assemble into complexes that mediate different fusion events. Assembly of distinct SNARE protein complexes is regulated by cognate SM proteins. Of the three exocytic SM proteins (Munc18a, b, c), the least is known about Munc18b, which preferentially binds Syn-2 and Syn-3. We have examined the role of Munc18b, intrinsically present in the insulin granules of a pancreatic islet beta-cell (FIG. 1). Based on a computational model of Munc18b-Syntaxin 3 complex (FIG. 2), we constructed Munc18b mutants (red circles) that have distinct binding affinities to cognate Syn-2 and Syn-3, which either reduced (Munc18b-E59) or increased assembly with SNARE complexes formed with Syn-2 and Syn-3 (FIG. 3, red box). The Munc18b-K314L/R315L (KR) mutant and to lesser extent, Munc18b wild type (WT), were found to facilitate SNARE complex assembly (FIG. 3, red box) and this greatly amplified biphasic glucose-stimulated insulin secretion (Fig, 4), but more so by the KR mutant. Remarkably, this Munc18b-mediated improvement of secretory efficiency was due to promotion in the frequency, extent and kinetics of insulin granule-granule fusion that was highest in the KR mutant (E.M. in FIG. 5), less so with WT, and greatly reduced in the E59 mutant.

The Invention: These results led us to believe that granule-granule fusion per se is mediated primarily by Munc18b-Syn-3 (and probably also Syn-2), and which is greatly facilitated by Munc18b-KR which induces a more ‘activated’ Syn-3 (and also Syn-2) SNARE complex (with SNAP25 (and SNAP-23) and VAMP (VAMP2 and VAMP8) that favors granule-granule fusion (FIG. 6) in many cell types that contain these exocytotic proteins. Enhancing the actions of Munc18b-SNARE complexes by employing Munc18b-KR mutants (K314L/R315L), peptides or mimicking compounds, to mobilizing more granules to the plasma membrane and to undergo sequential granule-granule fusion could be a treatment for diseases with underlying basis to be exocytotic defects (Application 1). Conversely, disrupting Munc18b-SNARE complex could reduce granule-granule fusion for treatment of diseases with underlying basis of excessive granule-granule fusion (Application 2).

Application 1: Enhancing Munc18b-SNARE complex actions (FIG. 6) can increase exocytotic efficiency of the fewer surviving beta-cells in late stage diabetes, leading to increased release of insulin to attain normoglycemic control. These drug compounds could be used to potentiate the actions of current treatments (glucagon-like peptide 1 (GLP-1)-mimetics) used to treat diabetes, allowing reduced dosages that would in turn reduce acute and complications on other GLP-1 affected organs, and increase specific drug actions on the pancreatic islet beta-cell. Another possibility is to transduce expression of Munc18b-WT or KR by transient or permanent gene transfer into primary tissue (pancreatic islets) for transplantation, which would allow reduced islet mass required for transplantation, currently the major limiting factor for this modality of treatment of diabetes. A similar strategy to increase secretory efficiency is also applicable to other diseases of reduced secretory efficiency that contain Munc18b and SNARE proteins such as in chronic pancreatitis exhibiting exocrine insufficiency.

Application 2:

Compounds that block or disrupt Munc18b-SNARE complex formation, including mutants like or mimic Munc18b-E59 (E59K), would reduce granule-granule fusion, which can be employed on diseases of enhanced secretion such as of mast cells that release massive amount of histamine in allergic and anaphylactic reactions, skin eczemas and asthma. Other cell types that also contain these exocytotic proteins include platelets and gastric parietal cells (secrete acid), whereby drug molecules that disrupt Munc18b-SNARE complex would have therapeutic effects of anti-platelet (to treat cardiovascular disease, stroke) and reduced acid secretion (to treat peptic ulcer disease and gastroesophageal acid reflux disease), respectively.

Application 3:

Since the Munc18b-KR (K314, R315) site is conserved in Munc18a (K314, R315)) and Munc18c (K315, K316), and Munc18K-E59 site is also conserved in Munc18a (E59) and Munc18c (E63), similar approaches indicated above will also be expected to promote (Application 1) or block (Application 2) Munc18a-SNARE complex (Syntaxin 1A, SNAP-25, VAMP2) and Munc18c-SNARE complex (Syntaxin 4, SNAP23, VAMP2) formation, thus influencing many additional cell types that use these SM-SNARE complexes for their distinct exocytotic fusions. We are therefore also extending our claims to:

1. Munc18a and its respective cell types—neurons (regulation of neurotransmitter release) and neuroendocrine cells (regulation of hormone secretion) and others; and diseases that could be afflicted by a deficiency or excess of secretion in these cell types.

2. Munc18c and its respective cell types—adipose or fat tissues and muscle (regulation of glucose transporter exocytosis) and others; and diseases that could be afflicted by a deficiency (i.e. type 2 diabetes) or excess of secretion in these cell types.

### BRIEF DESCRIPTION OF THE DRAWINGS

The invention will be described in relation to the following drawings.

FIG. 1 shows a confocal microscopy image of a rat pancreatic islet beta cell wherein the insulin is co-localized with Munc18b, indicated by arrows on the Merge image; thus indicating that Munc18b is abundant in insulin granules.

FIG. 2 shows a computational model of Munc18b-Syntaxin 3 complex. The amino acids contained within the circles indicate the E59 and K314/R315 positions where we had introduced mutations.

FIG. 3 shows the immunoprecipitation (IP) by anti-Syntaxin-2 and anti-Syntaxin-3 antibodies of insulinoma cells (INS-1) transfected with different Munc18b mutants (subcloned into adenovirus or Ad). Within the box indicates that Munc18b-WT (wild type) and Munc18b-KR were co-precipitated by the Syn-2 and Syn-3 antibodies, but not the Munc18b-E59K mutant or the GFP (green fluorescent protein control)-transfected cells. The other SNARE proteins (VAMP2 and SNAP25) were also co-precipitated.

FIG. 4 shows that rat pancreatic islets loaded up in a chamber perifused with high glucose (16.7 mM) stimulated insulin release (measured by radioimmunoassay) in a biphasic manner (initial tall peak followed by a lower sustained level of release). Here, Ad-Munc18b-KR transfected islets exhibited much higher biphasic insulin secretion, followed by Ad-Munc18b-WT-transfected islets, and both were much more than Ad-Munc18b-E59K transfected islets and the control.

FIG. 5 shows an electron microscopy image of rat pancreatic islet beta cells transfected with the Ad-Munc18b-KR showing a two-granule fusion with the lead granule forming a pore with the plasma membrane (indicated by arrow). This structure is much more common in Munc18b-KR, followed by Munc18b-WT, and then control, and the least in Munc18b-E59K transfected is let beta cells.

FIG. 6 shows a model that explains our hypothesis. Granule-granule fusion is mediated by Munc18b-Syn-3 (and probably also Syn-2) complex which enables further SNARE complex formation with SNAP25 and VAMPs (VAMP2 and VAMP8) in many cell types that contain these exocytotic proteins. Munc18b-KR mutant greatly facilitates this process while Munc18b-E59K blocks this process.

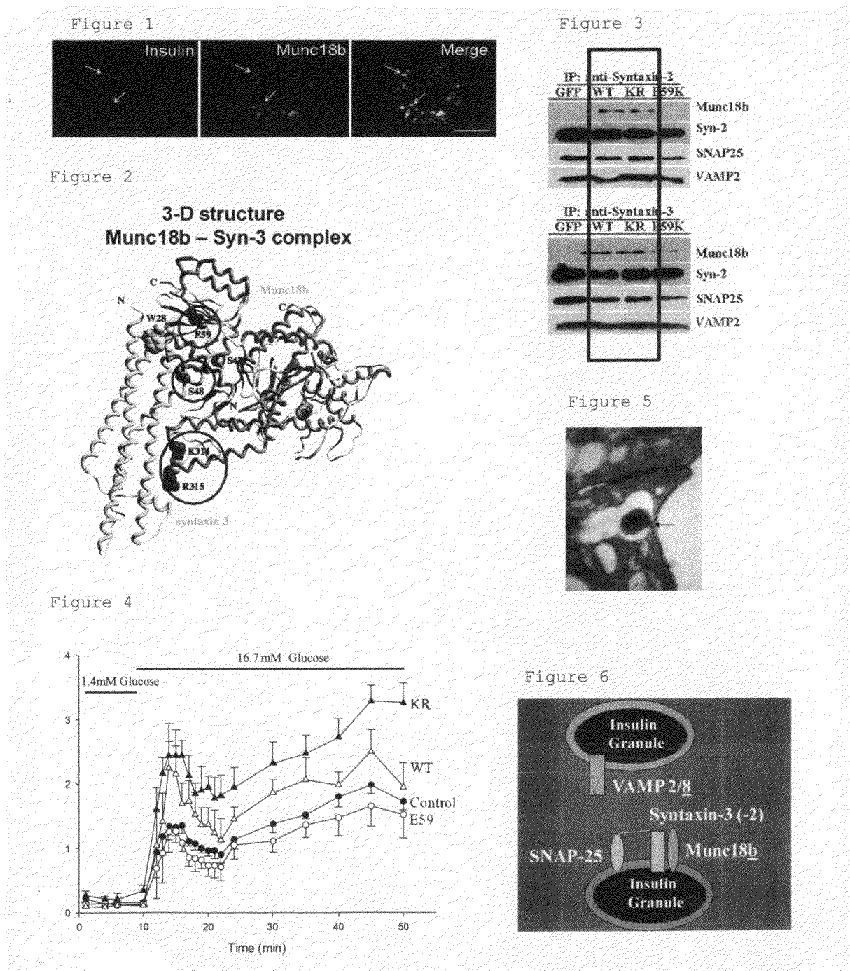

We claim:

1. The compounds that mimic the mutation within the protein Munc18b, Munc18b K314L/R315L (Munc18b-KR) in whole or in part, whose biochemical action is to enhance complex formation with Syntaxins (Syntaxin-2 and Syntaxin-3 primarily, and also Syntaxin-1) in a manner that induces SNARE complex assembly (with the families of VAMP and SNAP-25 proteins); where such ‘activated’ complexes function would promote membrane fusion between two secretory vesicles, which would potentiate secretion in all secretory cells that employ vesicle-vesicle fusion.

2. The compounds that mimic the mutation within the Munc18b protein, Munc18b E59K in whole or in part, whose biochemical action is to reduce complex formation with Syntaxins (Syntaxin-2 and Syntaxin-3 primarily, and also Syntaxin 1), in a manner that reduces SNARE complex assembly (with the families of VAMP and SNAP-25 proteins); and this would functionally abrogate membrane fusion between two vesicles, which would reduce secretion in all secretory cells that employ vesicle-vesicle fusion.

3. The methods for screening such compounds in claim 1 and claim 2, which affect the binding of the full length protein or a fragment of Munc18b-KR (in claim 1) or Munc18b E59 (in claim 2), with full length or fragments of the Syntaxins (Syntaxin-2, Syntaxin-3 and Syntaxin-1).

4. The compounds according to claim 1, wherein the Munc18b-KR mutation is conserved in the other members of the Munc18 family of proteins, and these Muncl8 proteins selected from the group include Munc18a (K314, R315) and Munc18c (K315, K316). Munc18a interacts with Syntaxin 1 primarily, and also with Syntaxin-2 and Syntaxin-3; whereas Munc18c interacts with Syntaxin-4 primarily, and also with Syntaxin-2. These interactions of Munc18a and Munc18c with their respective Syntaxins can promote membrane fusion processes in secretory cells other than vesicle-vesicle fusion which will promote secretion.

5. The compounds according to claim 2, wherein Munc18b-E59K mutation is conserved in the other members of the Munc18 family of proteins, and these Munc18 proteins selected from the group include Munc18a (E59) and Munc18c (E63). Munc18a interacts with Syntaxin 1 primarily, and also with Syntaxin-2 and Syntaxin-3; whereas Munc18c interacts with Syntaxin-4 primarily, and also with Syntaxin-2. These interactions of Munc18a and Munc18c with their respective Syntaxins can block membrane fusion processes in secretory cells other than vesicle-vesicle fusion which will inhibit secretion.

6. The methods according to claim 3 (Munc18b) for screening such compounds in claim 4 (KR mutants) and claim 5 (E59 mutants) between the other members of the Munc18 family (Munc18a and Munc18c) and their respective Syntaxin binding partners (Syntaxin-1, Syntaxin-2, Syntaxin-3 and Syntaxin-4), in either full length or fragments of these proteins (Munc18 and Syntaxin).

7. The usage of compounds in claim 1 and claim 4 in combination with any existing or future drugs or compounds that promote secretion.

8. The usage of compounds in claim 2 and claim 5 in combination with any existing or future drugs or compounds that block secretion.
